(19) United States
(12) Patent Application Publication
(10) Pub. No.: US 2004/0248939 A1
(43) Pub. Date: Dec. 9, 2004

(54) Stable pharmaceutical compositions comprising acid labile benzimidazoles

(76) Inventors: Masae Sugaya, Osaka (JP); Toshihiro Shimizu, Hyogo (JP)

Correspondence Address: WENDEROTH, LIND & PONACK, L.L.P., 2033 K STREET N. W., SUITE 800, WASHINGTON, DC, 20006-1021, US

(21) Appl. No.: 10/487,809

(22) PCT Filed: Aug. 29, 2002
(86) PCT No.: PCT/JP02/08704

(30) Foreign Application Priority Data
Aug. 31, 2001 (JP) 2001263481
Nov. 7, 2001 (JP) 2001341477
Mar. 6, 2002 (JP) 2002060006

(51) Int. Cl. A61K 31/4439; A61K 9/20

(52) U.S. Cl. 514/338; 424/465

(57) ABSTRACT

This invention provides a solid preparation without enteric coating which contains an acid labile active ingredient, particularly, a benzimidazole compound having an antiulcer action, and can neutralize the acid in stomach quickly, and exerts quickly the pharmacological effect of the active ingredient and suppresses the generation of a carbon dioxide gas as much as possible.

A gastric disintegrable solid preparation contains an acid labile active ingredient, particularly, a benzimidazole compound, and at least one component selected from metal oxides and metal hydroxides. The preparation does not enteric-coated, but has a disintegration time of 7 minutes or less.

### TECHNICAL FIELD

The present invention relates to a solid preparation, further in detail, to a medical solid preparation containing an acid labile active ingredient, particularly, an acid labile active ingredient such as a benzimidazole compound useful as an antiulcer agent.

### BACKGROUND ART

Benzimidazole compounds such as lansoprazole, omeprazole, rabeprazole and the like are widely used as a digestive ulcer therapeutic agent because of its gastric acid secretion suppressing action and gastric mucous membrane preventing action and the like.

However, these compounds have poor stability, and unstable to humidity, temperature and light. They are particularly unstable to an acid, and become extremely unstable in aqueous solution or suspension as the pH of the solution or suspension lowers.

In a preparation, namely, a tablet, powder, fine particles, capsule and the like, benzimidazole compounds become unstable since mutual interaction with other components of the preparation is stronger in a preparation than that of the compounds alone, and consequently, coloration change or decomposition is observed in production and storage. For stabilization of them, JP-A 10-36290 discloses enteric granules or enteric fine particles obtained by compounding a stabilizer composed of an inorganic base salt of magnesium and/or calcium for a medical solid composition, then, applying an enteric coating.

However, for producing such an enteric preparation, a process is required in which fine particles or granules containing a benzimidazole compound are produced, then, an enteric coating is applied. Further, since it takes a longer time until an enteric film is dissolved and a medicine is absorbed in a digestive tract after administration, a quick pharmacological effect can not be expected in the early stages after administration.

On the other hand, U.S. Pat. No. 5,840,737 and WO 00/26185 disclose a solution, suspension, tablet and capsule obtained by combining omeprazole or lansoprazole, which is not enteric-coated, with an alkali metal salt of bicarbonate.

However, since these preparations are combined with a bicarbonate, they react with an acid in stomach to evolve carbon dioxide gas which causes burping, and therefore they are not preferable from the viewpoint of compliance.

### OBJECTS OF THE INVENTION

An object of the present invention is to provide a solid preparation having no enteric coating which is capable of neutralizing quickly an acid in stomach, realizing quick occurrence of pharmacological effect of an active ingredient, and suppressing the evolution of carbon dioxide gas as much as possible, by solving the above-mentioned problems in medical solid preparations containing an acid labile active ingredient typically including benzimidazole compounds.

### SUMMARY OF THE INVENTION

The present inventors have found that a metal oxide and/or metal hydroxide is suitable for a gastric acid neutralizing agent in a solid preparation containing an acid labile active ingredient and having no enteric coating, and further investigation resulted in completion of the present invention.

Namely, the present invention provides:

(1) A gastric disintegrable solid preparation comprising an acid labile active ingredient and at least one component selected from metal oxides and metal hydroxides;

(2) A solid preparation according to the above-mentioned (1), wherein the disintegration time is within 7 minutes;

(3) A solid preparation according to the above-mentioned (1), which is the preparation without enteric coating;

(4) A solid preparation according to the above-mentioned (1), which comprises further at least one component selected from carbonates of alkali earth metal and basic additives having high water-solubility;

(5) A solid preparation according to the above-mentioned (1), wherein an acid labile active ingredient is a proton pump inhibitor (hereinafter, referred to as “PPI”);

(6) A solid preparation according to the above-mentioned (5), wherein the PPI is a benzimidazole compound;

(7) A solid preparation according to the above-mentioned (6), wherein a benzimidazole compound is a compound represented by the formula (I):

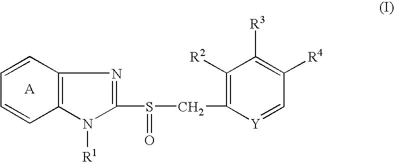

wherein ring A is an optionally substituted benzene ring, R¹ is hydrogen atom, an optionally substituted aralkyl group, acyl group or acyloxy group, R², R³ and R⁴ are the same or different and each represent a hydrogen atom, an optionally substituted alkyl group, an optionally substituted alkoxy group or an optionally substituted amino group, and Y represents a nitrogen atom or CH, or a salt thereof;

(8) A solid preparation according to the above-mentioned (6), wherein a benzimidazole compound is lansoprazole, omeprazole, rabeprazole or pantoprazole, or an optically active compound thereof;

(9) A solid preparation according to the above-mentioned (1), wherein the metal oxides and the metal hydroxides are those of which 1% aqueous solution or 1% aqueous suspension has a pH of 8.0 or more;

(10) A solid preparation according to the above-mentioned (1) which comprises at least one metal oxide selected from the group consisting of magnesium oxide, magnesium silicate, dry aluminum hydroxide gel and magnesium metasilicate aluminate;

(11) A solid preparation according to the above-mentioned (1) which comprises at least one metal hydroxide selected from the group consisting of magnesium hydroxide, aluminum hydroxide, synthetic Hydrotalcite, coprecipitate of aluminum hydroxide and magnesium hydroxide, coprecipitate of aluminum hydroxide, magnesium carbonate and calcium carbonate, and coprecipitate of aluminum hydroxide and sodium bicarbonate;

(12) A solid preparation according to the above-mentioned (4), wherein the carbonate of alkali earth metal is calcium carbonate or magnesium carbonate;

(13) A solid preparation according to the above-mentioned (4), wherein the basic additive having high water-solubility is trometamol, disodium succinate, sodium hydrogen phosphate, trisodium phosphate, dipotassium phosphate or L-arginine;

(14) A solid preparation according to the above-mentioned (1) which contains magnesium oxide;

(15) A solid preparation according to the above-mentioned (1) which contains magnesium hydroxide;

(16) A solid preparation according to the above-mentioned (1) which contains magnesium oxide and magnesium hydroxide;

(17) A solid preparation according to the above-mentioned (14) or (16), wherein the magnesium oxide is one obtained by calcination at a temperature ranging from about 500° C. to about 1000° C. and of purity higher than 95%;

(18) A solid preparation according to the above-mentioned (14), wherein the magnesium oxide has a BET specific surface area of about 10 m²/g to about 50 m²/g.

(19) A solid preparation according to the above-mentioned (6), which contains at least one component selected from metal oxides and metal hydroxides at a ratio of 0.1 to 1500 parts by weight relative to 1 part by weight of the benzimidazole compound;

(20) A solid preparation according to the above-mentioned (6), which contains at least one component selected from metal oxides and metal hydroxides together with a salt of alkali earth metal at a total ratio thereof of 0.1 to 1800 parts by weight relative to 1 part by weight of the benzimidazole compound;

(21) A solid preparation according to the above-mentioned (1), which is a tablet, a granule or a capsule;

(22) A solid preparation according to the above-mentioned (1), wherein a group containing an acid labile active ingredient and a group containing a metal oxide or a metal hydroxide but containing no active ingredient are separately compounded; and

(23) A solid preparation according to the above-mentioned (4), wherein (1) a group containing both an active ingredient and at least one component selected from metal oxides, metal hydroxides, carbonates of alkali earth metal and basic additives having high water-solubility and (2) a group not containing an acid labile active ingredient but containing at least one component selected from metal oxides, metal hydroxides, carbonates of alkali earth metal and basic additives having high water-solubility are separately compounded.

### DETAILED DESCRIPTION OF THE INVENTION

The acid labile active ingredient in the present invention is not particularly restricted, and any active components becoming unstable when exposed to gastric acid can be applied. Examples of the acid labile active ingredient include PPIs, erythromycin antibacterial compounds, anti-inflammatory enzymatic agents such as serrapeptase, semialkali proteinase and the like. Particularly, the present invention is suitable for PPIs. Such PPIs include benzimidazole compounds and similar compounds such as imidazopyridine compounds, e.g. tenatoprazole. Examples of benzimidazole compounds will be described below, however, the present invention is not limited to them and can be also applied to other active components unstable to an acid.

The benzimidazole compound which is a PPI, used in the present invention, includes a compound represented by the formula (I):

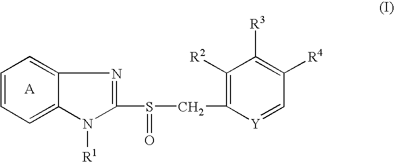

wherein, ring A represents an optionally substituted benzene ring, R¹ represents a hydrogen atom, an optionally substituted aralkyl group, acyl group or acyloxy group, R², R³ and R4 are the same or different and each represent a hydrogen atom, an optionally substituted alkyl group, an optionally substituted alkoxy group or an optionally substituted amino group, and Y represents a nitrogen atom or CH, or a salt thereof.

In the formula (I), the compound is preferably a compound wherein ring A is a benzene ring which may optionally have a substituent group selected from a halogen atom, an optionally halogenated C₁₋₄ alkyl group, an optionally halogenated C₁₋₄ alkoxy group and 5 or 6-membered heterocyclic group, R¹ is a hydrogen atom, R² is a C₁₋₆ alkyl group, C₁₋₆ alkoxy group, C₁₋₆ alkoxy-C₁₋₆ alkoxy group or di-C₁₋₆ alkylamino group, R³ is a hydrogen atom, C₁₋₆ alkoxy-C₁₋₆ alkoxy group or optionally halogenated C₁₋₆ alkoxy group, R⁴ is a hydrogen atom or C₁₋₆ alkyl group, and Y is a nitrogen atom.

Particularly preferable is the compound represented by the formula (Ia):

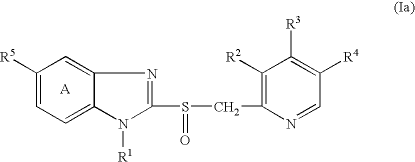

wherein, R¹ is a hydrogen atom, R² is a C₁₋₃ alkyl group or C₁₋₃ alkoxy group, R³ is a C₁₋₃ alkoxy group which may be halogenated or substituted by C₁₋₃ alkoxy group, R⁴ is a hydrogen atom or C₁₋₃ alkyl group, and R⁵ is a hydrogen atom, optionally halogenated C₁₋₃ alkoxy group or pyrrolyl group (e.g., 1-, 2- or 3-pyrrolyl group).

In the formula (Ia), particularly preferable is the compound wherein R¹ is a hydrogen atom, R² is a C₁₋₃ alkyl group, R³ is an optionally halogenated C₁₋₃ alkoxy group, R⁴ is a hydrogen atom, and R⁵ is a hydrogen atom or an optionally halogenated C₁₋₃ alkoxy group.

In the compound represented by the formula (I) above (hereinafter, referred to as compound (I)), the “substituent groups” in “an optionally substituted benzene ring” represented by ring A include, for example, a halogen atom, cyano group, nitro group, an optionally substituted alkyl groups, hydroxyl group, optionally substituted alkoxy group, aryl group, aryloxy group, carboxyl group, acyl group, acyloxy group, 5 to 10-membered heterocyclic group and the like, and 1 to 3 of these substituent groups may be substituted on a benzene ring. When the number of substituent groups is 2 or more, each substituent groups may be the same or different. Among these substituents, a halogen atom, an optionally substituted alkyl group and an optionally substituted alkoxy group are preferable.

As the halogen atom, a fluorine atom, chlorine atom, bromine atom and the like are exemplified, among which a fluorine atom is preferable.

Examples of “alkyl group” in “an optionally substituted alkyl group” include C₁₋₇ alkyl group (for example, methyl, ethyl, propyl, isopropyl, butyl, isobutyl, sec-butyl, tert-butyl, pentyl, hexyl, heptyl and the like). Examples of “substituent group” in “an optionally substituted alkyl group” include a halogen atom, hydroxy group, C₁₋₆ alkoxy group (for example, methoxy, ethoxy, propoxy, butoxy, etc.), C₁₋₆ alkoxy-carbonyl group (for example, methoxycarbonyl, ethoxycarbonyl, propoxycarbonyl, etc.), carbamoyl group and the like, and the number of these substituent groups may be 1 to 3. When the number of substituent groups is 2 or more, each substituent groups may be the same or different.

Examples of “alkoxy group” in “an optionally substituted alkoxy group” include C₁₋₆ alkoxy group (for example, methoxy, ethoxy, propoxy, isopropoxy, butoxy, isobutoxy, pentoxy, etc.). Examples of “substituent group”in “an optionally substituted alkoxy group” include groups identical with the “substituent group” of the “optionally substituted alkyl group” described above, and the number of substituent groups is also the same as that of the “optionally substituted alkyl group”.

The “aryl group” includes, for example, C₆₋₁₄ aryl group (e.g., phenyl, 1-naphtyl, 2-naphthyl, biphenyl, 2-anthryl, etc.) and the like.

The “aryloxy group” includes, for example, C₆₋₁₄ aryloxy group (e.g., phenyloxy, 1-naphtyloxy, 2-naphthyloxy, etc.) and the like.

The “acyl group” includes, for example, formyl, alkylcarbonyl, alkoxycarbony, carbamoyl, alkylcarbamoyl, alkylsulfinyl, alkylsulfonyl and the like.

The “alkylcarbonyl group” includes, for example, C₁₋₆ alkyl-carbonyl group (e.g., acetyl, propionyl, etc.) and the like.

The “alkoxycarbonyl group” includes, for example, C₁₋₆ alkoxy-carbonyl group (e.g., methoxycarbonyl, ethoxycarbonyl, propoxycarbonyl, butoxycarbonyl, etc.) and the like.

The “alkylcarbamoyl group” includes N-C₁₋₆ alkyl-carbamoyl group (e.g., methylcarbamoyl, ethylcarbamoyl, etc.), N,N-diC₁₋₆ alkyl-carbamoyl group (e.g., N,N-dimethylcarbamoyl, N,N-diethylcarbamoyl, etc.) and the like.

The “alkylsulfinyl group” includes, for example, C₁₋₇ alkylsulfinyl group (e.g., methylsulfinyl, ethylsulfinyl, propylsulfinyl, isopropylsulfinyl, etc.) and the like.

The “alkylsulfonyl group” includes, for example, C₁₋₇ alkylsulfonyl group (e.g., methylsulfonyl, ethylsulfonyl, propylsulfonyl, isopropylsulfonyl, etc.) and the like.

The “acyloxy group” includes, for example, alkylcarbonyloxy group, alkoxycarbonyloxy group, carbamoyloxy group, alkylcarbamoyloxy group, alkylsulfinyloxy group, alkylsulfonyloxy group and the like.

The “alkylcarbonyloxy group” includes C₁₋₆ alkyl-carbonyloxy group (e.g., acetyloxy, propionyloxy, etc.) and the like.

The “alkoxycarbonyloxy group” includes, for example, C₁₋₆ alkoxy-carbonyloxy group (e.g., methoxycarbonyloxy, ethoxycarbonyloxy, propoxycarbonyloxy, butoxycarbonyloxy, etc.) and the like.

The “alkylcarbamoyloxy group” includes C₁₋₆ alkyl-carbamoyloxy group (e.g., methylcarbamoyloxy, ethylcarbamoyloxy, etc.) and the like.

The “alkylsulfinyloxy group” includes, for example, C₁₋₇ alkyl-sulfinyloxy group (e.g., methylsulfinyloxy, ethylsulfinyloxy, propylsulfinyloxy, isopropylsulfinyloxy, etc.) and the like.

The “alkylsulfonyloxy group” includes, for example, C₁₋₇ alkyl-sulfonyloxy group (e.g., methylsulfonyloxy, ethylsulfonyloxy, propylsulfonyloxy, isopropylsulfonyloxy, etc.) and the like.

The “5 to 10-membered heterocyclic group” includes, for example, 5 to 10-membered (preferably, 5 or 6-membered) heterocyclic group having 1 or more (for example, 1 to 3) hetero atoms selected from a nitrogen atom, sulfur atom and oxygen atom in addition to a carbon atom, and specific examples thereof include 2- or 3-thienyl group, 2-, 3- or 4-pyridyl group, 2- or 3-furyl group, 1-, 2- or 3-pyrrolyl group, 2-, 3-, 4-, 5- or 8-quinolyl group, 1-, 3-, 4- or 5-isoquinolyl group, 1-, 2- or 3-indolyl group and the like. Among them, preferable are 5 or 6-membered heterocyclic group such as 1-, 2- or 3-pyrrolyl group.

Preferably, ring A is a benzene ring which may have one or two substituent groups selected from a-halogen atom, an optionally halogenated C₁₋₄ alkyl group, an optionally halogenated C₁₋₄ alkoxy groups and 5 or 6-membered heterocyclic group.

Examples of “aralkyl group” in “an optionally substituted aralkyl group” represented by R¹ include, for example, C₇₋₁₆ aralkyl group (e.g., C₆₋₁₀ aryl C₁₋₆ alkyl group such as benzyl, phenetyl, etc.) and the like. Examples of “substituent group” in “an optionally substituted aralkyl group” include the same substituent groups as those of the “optionally substituted alkyl group” described above, and the number of substituent groups is 1 to 4. When the number of substituent groups is 2 or more, each substituent groups may be the same or different.

The “acyl group” represented by R¹ includes, for example, the “acyl group” exemplified as the substituent group on ring A described above.

The “acyloxy group” represented by R¹ includes, for example, the “acyloxy group” exemplified as the substituent group on ring A described above.

Preferably, R¹ is a hydrogen atom.

The “optionally substituted alkyl group” represented by R², R³ or R⁴ includes the “optionally substituted alkyl group” exemplified as the substituent group on ring A described above.

The “optionally substituted alkoxy group” represented by R², R³ or R⁴ includes the “optionally substituted alkoxy group” exemplified as the substituent group on ring A described above.

The “optionally substituted amino group” represented by R², R³ or R⁴ includes, for example, amino group, mono-C₁₋₆ alkylamino group (e.g., methylamino, ethylamino, etc.), mono-C₆₋₁₄ arylamino group (e.g., phenylamino, 1-naphthylamino, 2-naphthylamino, etc.), di-C₁₋₆ alkylamino group (e.g., dimethylamino, diethylamino, etc.), di-C₆₋₁₄ arylamino group (e.g., diphenylamino, etc.) and the like.

Preferably, R² is a C₁₋₆ alkyl group, C₁₋₆ alkoxy group, C₁₋₆ alkoxy-C₁₋₆ alkoxy group or -di-C₁₋₆ alkylamino group. More preferably, R² is a C₁₋₃ alkyl group or C₁₋₃ alkoxy group.

Preferably, R³ is a hydrogen atom, C₁₋₆ alkoxy-C₁₋₆ alkoxy group or optionally halogenated C₁₋₆ alkoxy group. More preferably, R³ is a C₁₋₃ alkoxy group which is halogenated or may be substituted with a C₁₋₃ alkoxy group.

Preferably, R⁴ is a hydrogen atom or C₁₋₆ alkyl group. More preferably, R⁴ is a hydrogen atom or C₁₋₃ alkyl group (particularly, hydrogen atom).

Preferably, Y is a nitrogen atom.

Specific examples of the compound (I) include the following compounds.

2-[[[3-methyl-4-(2,2,2-trifluoroethoxy)-2-pyridinyl]methyl]sulfinyl]-1H-benzimidazole, 2-[[3,5-dimethyl-4-methoxy-2-pyridinyl)methyl]sulfinyl]-5-methoxy-1H-benzimidazole, 2-[[[4-(3-methoxypropoxy)-3-methyl-2-pyridinyl]methyl]sulfinyl]-1H-benzimidazole.sodium salt, 5-difluoromethoxy-2-[[(3,4-dimethoxy-2-pyridinyl)methyl]sulfinyl]-1H-benzimidazole and the like.

Among these compounds, 2-[[[3-methyl-4-(2,2,2-trifluoroethoxy)-2-pyridinyl]methyl]sulfinyl]-1H-benzimidazole(Lansoprazole) is preferable.

The above-mentioned compound (I) may be a racemic compound, or may be an optically active compound such as R-compound, S-compound and the like. For example, optically active substances such as (R)-2-[[[3-methyl-4-(2,2,2-trifluoroethoxy)-2-pyridinyl]methyl]sulfinyl]-1H-benzimidazole (sometimes referred to as Lansoprazole R enantiomer) may also be permissible and preferable.

The salt of the compound (I) is preferably a pharmaceutically acceptable salt, and examples thereof include salts with inorganic bases, salts with organic bases, salts with basic amino acids, and the like.

Suitable examples of the salt with an inorganic base include, for example, alkali metal salts such as sodium salts, potassium salts, etc.; alkaline earth metal salts such as calcium salts, magnesium salts, etc.; ammonium salts, and the like.

Suitable examples of the salt with an organic base include, for example, salts with alkylamines (trimethylamine, triethylamine, etc.), heterocyclic amines (pyridine, picoline, etc.), alkanolamines (ethanolamine, diethanolamine, triethanolamine, etc.), dicyclohexylamine, N,N′-dibenzylethylenediamine and the like.

Suitable examples of the salt with a basic amino acid include, for example, salts with alginine, lysine, ornithine and the like.

Among these salts, alkali metal salts or alkaline earth metal salts are preferable. Particularly, sodium salts are preferable.

The compound (I) can be produced by a method known per se, and produced by methods described, for example, JP-A 61-50978, U.S. Pat. No. 4,628,098, JP-A 10-195068, WO 98/21201 and the like, or methods according to these methods. The optically active compound (I) can be obtained by optical resolution methods (fractional re-crystallization method, chiral column method, diastereomer method, method using microorganism or enzyme, etc.), asymmetric oxidation and the like. For example, in the case of Lansoprazole R enantiomer, it can also be produced in accordance with the methods described in WO 00-78745, WO 01-83473, WO 01-87874 and WO 02-44167.

As the PPIs used in the present invention, the benzimidazole compound having an antiulcer action such as lansoprazole, omeprazole, rabeprazole and pantoprazole and the imidazopyridine compound such as tenatoprazole or optically active compounds thereof and pharmaceutically acceptable salts thereof are preferable.

The compounding amount of the benzimidazole compound used in the present invention varies depending on the kind and dosage of an active ingredient, and for example, the amount is from 0.001 to 0.3 parts by weight, preferably from 0.002 to 0.2 parts by weight relative to 1 part by weight of the solid preparation of the present invention.

The metal oxide and metal hydroxide used in the present invention are preferably those of which 1% aqueous solution or 1% aqueous suspension has a pH of 8.0 or more, and examples of the metal oxide include medical magnesium oxide, magnesium silicate (2MgO.3SiO₂.xH₂O), dry aluminum hydroxide gel (Al₂O₃. xH₂O), magnesium metasilicate aluminate (Al₂O₃.MgO.2SiO₂.xH₂O) and the like. Particularly, magnesium oxide can be suitably used.

Preferable magnesium oxides are those that are available for medical use and that have an excellent reactivity to acid and neutralization ability. As these magnesium oxides, magnesium oxide obtained by a usual production method and commercially available magnesium oxide can be used, and preferable is one obtained by calcination at low temperature, so-called, calcining magnesia. The magnesium oxide calcined at a temperature of about 500 to about 1000° C. is generally preferable, and particularly from the viewpoint of neutralization ability the magnesium oxide calcined at a temperature of about 600 to about 900° C. is preferable, and the magnesium oxide calcined at about 800° C. is most preferable. Among these magnesium oxides, favorable is the one that neutralizes the environment prior to the release of the acid labile active ingredient by the disintegration of the preparation in stomach and has the function to enhance the remaining ratio of the active ingredient. Such magnesium oxide is preferably the one that has usually a BET specific surface area of about 10 m²/g to about 50 m²/g, preferably about 20 m²/g to about 5 m²/g.

Hereupon, a BET specific surface area means the specific surface area measured by nitrogen gas adsorption method, and the specific surface area containing the surface of given amount magnesium oxide and its cavity in which nitrogen gas can enter is determined by the amount of adsorbed nitrogen gas.

The magnesium oxide includes, for example, commercially available heavy magnesium oxide (manufactured by Kyowa Kagaku Kogyo K.K.), heavy magnesium oxide (Tomita Pharmaceutical Co. Ltd.), heavy N magnesium oxide (manufactured by Kyowa Kagaku Kogyo K.K.), light magnesium oxide (manufactured by Kyowa Kagaku Kogyo K.K.) and the like. Particularly heavy N magnesium oxide (manufactured by Kyowa Kagaku Kogyo K.K.) is preferable.

The metal hydroxide includes, for example, medical magnesium hydroxide, aluminum hydroxide, synthetic hydrotalcite (Mg₆Al₂ (OH)₁₆CO₃.4H₂O), co-precipitate of aluminum hydroxide and magnesium hydroxide, co-precipitate of aluminum hydroxide, magnesium carbonate and calcium carbonate, and co-precipitate of aluminum hydroxide and sodium hydrogen carbonate. Among these compounds, magnesium hydroxide is particularly preferable from the viewpoint of the disintegrating property and dissolution property of a preparation.

These may be used alone or in combination of two or more. Some of metal oxides and metal hydroxides may whittle the surface of a preparation apparatus in production. As a result of such whittling, the resulting tablets sometimes become partially or wholly darkish or blackish and are imparted with black spots, lines or surfaces. Sticking of the resulting preparations on a die in production of tablets is also sometimes caused, depending on the metal hydroxides or metal oxides used. These properties deteriorate remarkably the productivity. It has been found that, when metal oxides or metal hydroxides having whittling property and-adhesiveness on a die are used, the whittling action and adhesiveness on a die can be suppressed by wet or dry granulation using metal oxides or metal hydroxides having no such properties or pharmaceutically acceptable additives described bellow (excipients, binders, disintegrants, etc.) in combination. In the case of preparations of PPIs, preferred are magnesium hydroxides, magnesium oxides and combination of a magnesium hydroxide and magnesium oxide from the viewpoint of compatibility with PPIs, dissolution property, and disintegrating property of a preparation.

These metal oxides and/or metal hydroxides are compounded in such an amount that they are quickly dissolved and neutralize gastric acid simultaneously with disintegration of a solid preparation in stomach, preferably, prior to dissolution of an active ingredient, in order to prevent unstabilization of substantial parts of an active ingredient by being exposed to gastric acid. Metal oxides and metal hydroxides are compounded usually in an amount of about 0.05 to 2000 parts by weight, preferably about 0.1 to 1000 parts by weight, more preferably about 0.1 to 800 parts by weight relative to 1 part by weight of an acid labile active ingredient, though the amount varies depending on the gastric acid neutralization ability of each metal oxide and metal hydroxide. For example, metal oxides and metal hydroxides are compounded in an amount of about 0.1 to 1500 parts by weight, preferably about 0.5 to 800 parts by weight, more preferably 0.1 to 400 parts by weight relative to 1 part by weight of a benzimidazole compound. When the active ingredient is a benzimidazole compound, the pH in stomach usually increases simultaneously with initiation of dosing, and they are compounded preferably in an amount that pH increases to 4 or more within about 60 minutes, more preferably within 40 minutes after administration, in stomach of usual pH range.

Usually, metal oxides and metal hydroxides are compounded preferably in an amount that pH increases to 7 or more within 10 minutes, more preferably within 7 minutes, by a measuring method as shown in the following experiment example.

In the present invention, at least one component selected from carbonates of alkaline earth metals and basic additives having high water-solubility may be compounded, in addition to these metal oxides and/or metal hydroxides, if necessary. The carbonates of alkaline earth metals include, for example, calcium carbonate and magnesium carbonate for medical use. The basic additives having high water-solubility include medical additives having an antacid action such as trometamol, disodium succinate, sodium hydrogen phosphate, trisodium phosphate, dipotassium phosphate, L-arginine and the like. These may also be used alone or in combination of two or more.

These are also compounded in such an amount that they are quickly dissolved and neutralize gastric acid simultaneously with disintegration of a solid preparation in stomach, preferably, prior to dissolution of an active ingredient, in order to prevent unstabilization of substantial parts of an active ingredient by being exposed to gastric acid, and are compounded usually in a total amount with metal oxides and metal hydroxides of about 0.05 to 2000 parts by weight, preferably about 0.1 to 1200 parts by weight, more preferably about 0.1 to 800 parts by weight relative to 1 part by weight of a acid labile active ingredient, though the amount varies depending on the gastric acid neutralization ability of each additives. Usually, neutralization agents are compounded in a total amount of 0.1 to 1800 parts by weight, preferably about 0.5 to 1000 parts by weight, more preferably 1 to 800 parts by weight relative to 1 part by weight of a benzimidazole compound. Preferably, they are compounded in an amount that pH increases to 4 or more within about 60 minutes, more preferably within 40 minutes after administration, in stomach of usual pH range.

In the solid preparation of the present invention, additives can be further used such as excipients for preparation (e.g., glucose, fructose, lactose, sucrose, D-mannitol, erythritol, maltitol, trehalose, sorbitol, corn starch, potato starch, wheat starch, rice starch, microcrystalline cellulose (crystalline cellulose), anhydrous silic acid, anhydrous calcium phosphate, precipitated calcium carbonate, calcium silicate, etc.), binder (e.g., hydroxypropylcellulose, hydroxypropylmethylcellulose, polyvinylpyrrolidone, methylcellulose, polyvinyl alcohol, carboxymethylcellulose sodium, partial a-starch, a-starch, sodium alginate, pullulan, gum Arabic powder, gelatin, etc.), disintegrating agent (e.g., low-substituted hydroxypropylcellulose, calmellose, calmellose calcium, carboxymethyl starch sodium, cross calmellose sodium, crospovidone, hydroxypropyl starch, etc.), flavoring agent (e.g., citric acid, ascorbic acid, tartaric acid, malic acid, aspartame, acesulfam potassium, somatin, saccharin sodium, dipotasium glycirrhizinate, sodium glutamate, sodium 5′-inosinate, sodium 5′-guanylate, etc), surfactant (e.g., polysorbate, polyoxyethylene.polyoxypropylene copolymer, sodium laurylsulfate, etc.), aromatics (e.g., lemon oil, orange oil, menthol, peppermint oil, etc.), lubricant (e.g., magnesium stearate, sucrose fatty acid ester, stearyl sodium fumarate, stearic acid, talc, polyethylene glycol, etc.), coloring agent (e.g., edible yellow No. 5, edible blue No. 2, ferric oxide, yellow ferric oxide, etc.) and antioxidant (e.g., sodium ascorbate, L-cysteine, sodium sulfite, etc.).

The particle size of a raw material used in them is not particularly restricted, and preferably 500 μm or less from the standpoint of a production property and dosing property.

The method of producing the solid preparation of the present invention may be a method known per se, and for example, benzimidazole compounds, metal oxides and/or metal hydroxides, if necessary, carbonates of alkaline earth metals and/or basic additives having higher water-solubility and an antacid action, excipients, further, binders, disintegrating agents, lubricants, flavoring agents, coloring agents, aromatics are combined suitably to give a tablet, powder, granule, capsule, fine particles and the like. These can be produced by a method described in the preparation general rule of The Pharmacopoeia of Japan, 14th revision.

Particularly, the granulation by wet granulation is preferred.

Herein, the wet granulation means a method for obtaining granulated materials or powders such as granules and fine granules by granulating a dispersion or solution of the mixture of a drug and excipient in water, binder or solvent and then drying, and the granulation mechanism may be any type such as extrusion, fluidization, rolling, centrifuging, stirring, spraying etc.

Further, these preparations may be coated with a coating agent (for example, coating film containing hydroxypropylmethylcellulose, hydroxypropylcellulose, polyvinyl alcohol, polyvinyl pyrrolidone, etc.), however, an enteric coating is not applied.

In the present invention, preparation raw materials may be formulated in one portion, or may be divided into two or more groups and formulated (for example, layer separation, granulations having different disintegrating properties, etc.). In any case, metal oxides and/or metal hydroxides, further, carbonates of alkaline earth metals and/or basic additives having higher water-solubility and an antacid property are quickly dissolved and neutralize gastric acid simultaneously with disintegration of a solid preparation in stomach, preferably, prior to dissolution of an active ingredient, and prevent unstabilization of substantial parts of an active ingredient by being exposed to gastric acid. For example, a method in which a group containing an active ingredient is compounded near the nucleus of a preparation and a metal oxide and/or metal hydroxide is compounded in an outer layer of the preparation are exemplified.

Also in either case of one-group formulation or divided or separate-groups formulation, it is possible to neutralize gastric acid by compounding a basic additive having high water solubility and dissolving it quickly.

Further, by dividing preparation raw materials into a group containing an acid labile active ingredient and a group containing no active ingredient and compounding them separately in the preparation to give a time difference of disintegration of components, the group containing no active component can be formulated to disintegrate more quickly. A metal oxide and/or metal hydroxide may be compounded in both groups or in the group containing no active ingredient. Further, a carbonate of an alkaline earth metal and/or a basic additive having high water solubility and an antacid action may be compounded in either group or both groups.

Furthermore, a preparation containing a group which contains neither an active ingredient nor a metal oxide and metal hydroxide but contains mainly a carbonate of an alkaline earth metal and/or a basic additive having high water solubility and an antacid action, may also be formulated. Particularly, this preparation is suitable to increase the pH in stomach by dissolving this group more quickly.

Further, when the components are grouped and formulated separately, an additive having bonding ability to a group containing an active ingredient (e.g., hydroxypropylcellulose, hydroxypropylmethylcellulose, hydroxyethylcellulose, polyvinylpyrrolidone, methylcellulose, polyvinyl alcohol, carboxymethylcellulose sodium, partial α-sytarch, α-sytarch, sodium alginate, pullulan, gum Arabic powder, gelatin, polyethylene oxide, carboxymethylethylcellulose, carboxyvinyl polymer, ethylcellulose, ethyl acrylate.methyl methacrylate.trimethylammoniumethyl methacrylate copolymer, etc.) may be compounded to delay the dissolution of the active ingredient. Further, a group containing an active component may be coated to delay the dissolution with a component containing hydroxypropylmethylcellulose, hydroxypropylcellulose, polyvinyl alcohol, polyvinylpyrrolidone, ethylcellulose or ethyl acrylate.methyl methacrylate.trimethylammoniumethyl methacrylate copolymer.

More specifically, a tablet can be produced, for example, by several methods such that a benzimidazole compound, metal hydroxide, excipient, binder, disintegrating agent and lubricant are mixed and compressed directly into tablets; a benzimidazole compound, a metal hydroxide, excipient and additive having high water solubility and an antacid action are mixed, then, a binder is added to the mixture to form granules, and a disintegrating agent and lubricant are added to the granules, and then the resultant mixture is compressed into tablets; and a benzimidazole compound, a metal hydroxide and excipient are mixed, then, a binder is added to the mixture to obtain granules, and separately, a metal hydroxide, additive having high water solubility and an antacid action and excipent are mixed, then, a binder is added to the mixture to obtain granules, and these obtained granules, disintegrating agent and lubricant are mixed and compressed into tablets.

Further, in the case of production of two or more kinds of granules, it is also possible that one or more kinds of binders are added to a group containing a benzimidazole compound to suppress its dissolution.

Granules can be produced by an ordinary method. For example, granules can be produced by the same methods as the production methods of a tablet, or by an extrusion granulation method. For obtaining granules having higher sphericity and smaller particle size distribution, for example, nucleus-containing granules may be produced by a method described in JP-A 63-301816. Nucleus-containing granules are obtained by coating a powdery spray agent containing a benzimidazole compound having an antiulcer action, metal hydroxide, excipient, disintegrating agent and the like while spraying binding liquid such as hydroxypropylcellulose on a sugar nucleus. The nucleus granule includes, for example, Nonparell obtained by coating sucrose (75 parts by weight) with corn starch (25 parts by weight) by a method known per se, and spherical nucleus granules using crystalline cellulose, and further, the nucleus granule itself may be the active ingredient component mentioned above. The average particle size of the nucleus granule is generally 14 to 80 mesh.

In the case of a capsule, it can be obtained by filling with a simply mixed powder or the particles for a tablet or granule obtained above.

The solid preparation obtained in the present invention is a gastric disintegrable solid preparation without enteric coating having an disintegration time of 7 minutes or less, preferably 5 minutes or less, more preferably 4 minutes or less, by the measurement of disintegrating time based on the method described in United States Pharmacopoeia <701>Disintegration.

The solid preparation of the present invention can be itself administered orally. The solid preparation of the present invention can be taken in the form of liquid or semisolid by dispersing or dissolving it previously in water, juice, yoghurt and the like.

In the solid preparation of the present invention, when the active ingredient is, for example, a benzimidazole compound represented by the formula (I) such as lansoprazole and optically active compounds thereof, these compounds are useful as a medicine since they have excellent antiulcer action, gastric acid secretion-suppressing action, mucous membrane protecting action, anti-Helicobacter pylori action and the like, and have low toxicity. In this case, the solid preparation of the present invention can be orally administered to mammal animals (for example, human, monkey, sheep, horse, dog, cat, rabbit, rat, mouse, etc.), for the purpose of treating and preventing peptic ulcer (for example, gastric ulcer, duodenal ulcer, stomal ulcer, Zollinger-Ellison syndrome, etc.), gastritis, Gastroesophageal Reflux Diseases (GERD) e.g. reflux esophagitis, Symptomatic GERD, erosive esophagitis; NUD (Non Ulcer Dyspepsia), stomach cancer (including stomach cancer caused by promotion of production of interleukin-1β by gene polymorphism of interleukin-1), stomach MALT lymphoma and the like, removing Helicobacter pylori, suppression of upper digestive canal hemorrhage caused by peptic ulcer, acute stress ulcer, and hemorrhagic gastritis, suppressing upper digestive canal hemorrhage caused by invasive stress (stress caused by cerebral vascular disorder requiring major operation or intensive care needing intensive management after operation, head trauma, multi-organ disorder, wider range heat injury), treating and preventing ulcer ascribed to nonsteroidal anti-inflammatory agent; and treating and preventing gastric hyperacidity and ulcer by stress after operation. For removal of Helicobacter pylori, it is preferable to use the solid preparation and, penicillin antibiotics (e.g., amoxicillin) and erythromycin antibiotics (e.g., clarithromycin), together.

The preparation of this invention is especially applicable for GERD (e.g., Symptomatic GERD and erosive esophagitis).

The daily dose differs depending on severity of symptom, age, sex and body weight of the patient, period and interval of administration, kind of the active ingredient employed and the like, and is not particularly restricted, and for example, the solid preparation can be administered as an antiulcer agent to an adult (60 kg) at an oral daily dose of about 0.5 to 1500 mg/day, preferably about 5 to 150 mg/day as an active ingredient. These benzimidazole compound-containing preparations may be administered once or in two or three divided portions a day.

### EXAMPLES

Hereinafter, the present invention is further detailed by the following Examples, which are not intended to restrict the present invention.

### Example 1

### Production of Active Ingredient Group

240 g of lansoprazole, 1160 g of magnesium hydroxide, 616 g of D-mannitol and 264 g of corn starch were charged into a fluidized bed granulator, and 8% aqueous solution prepared by dissolving 120 g of hydroxypropylcellulose in 1380 g of purified water was sprayed, and these materials were granulated, and dried to obtain 2188 g of granules.

### Production of Outer Layer Group

870 g of magnesium hydroxide, 1107 g of D-mannitol and 474 g of corn starch were charged in a fluidized bed granulator, and 750 g of purified water was sprayed, and these materials were granulated, and dried to obtain 2199 g of granules.

300 g of a active ingredient group, 408.5 g of an outer layer group, 37.5 g of crospovidone and 11 g of magnesium stearate were mixed in a bag to obtain a mixture. The resultant mixture was compressed into tablets (750 mg per tablet) by a die having a 13 mmΦ flat bevel edge using tabletting machine. No darkishness by whittled powders or sticking of the mixture on the die was observed in the resulting tablets.

### Example 2

### Production of Active Ingredient Group

120 g of lansoprazole, 200 g of magnesium hydroxide, 580 g of D-mannitol and 240 g of corn starch were charged into a fluidized bed granulator, and 8% aqueous solution prepared by dissolving 60 g of hydroxypropylcellulose in 690 g of purified water was sprayed, and these materials were granulated, and dried to obtain 1161.1 g of granules.

### Production of Outer Layer Group

720 g of magnesium hydroxide, 259.5 g of D-mannitol, 225 g of microcrystalline cellulose (Ceolus KG-801) and 112.5 g of crospovidone were charged in a fluidized bed granulator, and 500 g of purified water was sprayed, and these materials were granulated, and dried to obtain 1138.8 g of granules.

300 g of a active ingredient group, 439 g of an outer layer group and 11 g of magnesium stearate were mixed in a bag to obtain a mixture. The resultant mixture was compressed into tablets (750 mg per tablet) by a die having a 13 mmΦ flat bevel edge using tabletting machine. No darkishness by whittled powders or sticking of the mixture on the die was observed in the resulting tablets.

### Example 3

### Production of Active Ingredient Group

120 g of lansoprazole, 580 g of magnesium hydroxide, 332 g of D-mannitol and 108 g of corn starch were charged into a fluidized bed granulator, and 8% aqueous solution prepared by dissolving 60 g of hydroxypropylcellulose in 690 g of purified water was sprayed, and these materials were granulated, and dried to obtain 982.1 g of granules.

### Production of Outer Layer Group

108.8 g of magnesium hydroxide, 453.8 g of trometamol, 52.5 g of D-mannitol, 127.5 g of microcrystalline cellulose (Ceolus KG-801) and 63.7 g of crospovidone were charged in a fluidized bed granulator, and 400 g of purified water was sprayed, and these materials were granulated, and dried to obtain 758.7 g of granules.

270 g of a active ingredient group, 483.8 g of an outer layer group and 11.2 g of magnesium stearate were mixed in a bag to obtain a mixture. The resultant mixture was compressed into tablets (850 mg per tablet) by a die having a 13 mmΦ flat bevel edge using tabletting machine. No darkishness by whittled powders or sticking of the mixture on the die was observed in the resulting tablets.

### Example 4

150 g of lansoprazole, 500 g of magnesium oxide (manufactured by Kyowa Kagaku Kogyo K.K., grade: heavy N), 725 g of magnesium hydroxide, 1390 g of D-mannitol and 70 g of aspartame were charged into a fluidized bed granulator, and 2.8% aqueous solution prepared by dissolving 70 g of hydroxypropylcellulose in 2430 g of purified water was sprayed, and these materials were granulated, and dried to obtain 2771.5 g of granules.

2614.5 g of the obtained granules, 315 g of microcrystalline cellulose (Ceolus KG-801), 157.5 g of crospovidone and 63 g of magnesium stearate were mixed in a bag to obtain a mixture. The resultant mixture was compressed into tablets (700 mg per tablet) by a die having a 13 mmΦ flat bevel edge using tabletting machine. No darkishness by whittled powders or sticking of the mixture on the die was observed in the resulting tablets.

### Example 5

60 g of lansoprazole, 120 g of magnesium oxide, 406 g of magnesium hydroxide and 584 g of D-mannitol were charged into a fluidized bed granulator, and 5.6% aqueous solution prepared by dissolving 28 g of hydroxypropylcellulose in 472 g of purified water was sprayed, and these materials were granulated, and dried to obtain 1144.3 g of granules.

581 g of the granules, 70 g of microcrystalline cellulose (Ceolus KG-801), 35 g of crospovidone and 14 g of magnesium stearate were mixed in a bag to obtain a mixture. The resultant mixture was compressed into tablets (700 mg per tablet) by a die having a 13 mmΦ flat bevel edge using tabletting machine. No darkishness by whittled powders or sticking of the mixture on the die was observed in the resulting tablets.

### Example 6

150-g of lansoprazole, 500 g of magnesium oxide (manufactured by Kyowa Kagaku Kogyo K.K., N grade), 725 g of magnesium hydroxide, 1316.5 g of D-mannitol and 70 g of aspartame were charged into a fluidized bed granulator, and an aqueous solution prepared by dispersing and dissolving 140 g of hydroxypropylcellulose and 3.5 g of yellow ferric oxide in 2256.5 g of purified water was sprayed, and these materials were granulated, and dried to obtain 2817.7 g of granules.

2614.5 g of the granules, 315 g of microcrystalline cellulose (Ceolus KG-801), 157.5 g of crospovidone and 63 g of magnesium stearate were mixed in a bag to obtain a mixture. The resultant mixture was compressed into tablets (700 mg per tablet) by a die having a 13 mmΦ flat bevel edge using tabletting machine. No darkishness by whittled powders or sticking of the mixture on the die was observed in the resulting tablets.

### Example 7

105 g of lansoprazole, 525 g of magnesium oxide (manufactured by Kyowa Kagaku Kogyo K.K., N grade), 761.3 g of magnesium hydroxide, 1300.3 g of D-mannitol and 70 g of aspartame were charged into a fluidized bed granulator, and an aqueous solution prepared by dispersing and dissolving 140 g of hydroxypropylcellulose and 3.5 g of yellow ferric oxide in 2376.5 g of purified water was sprayed, and these materials were granulated, and dried to obtain 2754.6 g of granules.

2573 g of the granules, 310 g of microcrystalline cellulose (Ceolus KG-801), 155 g of crospovidone and 62 g of magnesium stearate were mixed in a bag to obtain a mixture. The resultant mixture was compressed into tablets (1000 mg per tablet) by a die having a 16 mmΦ flat bevel edge using tabletting machine. No darkishness by whittled powders or sticking of the mixture on the die was observed in the resulting tablets.

### Example 8

75 g of lansoprazole, 500 g of magnesium oxide (manufactured by Kyowa Kagaku Kogyo K.K., N grade), 725 g of magnesium hydroxide, 1391.5 g of D-mannitol and 70 g of aspartame were charged into a fluidized bed granulator, and an aqueous solution prepared by dispersing and dissolving 140 g of hydroxypropylcellulose, 1.75 g of yellow ferric oxide and 1.75 g of ferric oxide in 2256.5 g of purified water was sprayed, and these materials were granulated, and dried to obtain 2828.0 g of granules.

2614.5 g of the granules, 315 g of microcrystalline cellulose (Ceolus KG-801), 157.5 g of crospovidone and 63 g of magnesium stearate were mixed in a bag to obtain a mixture. The resultant mixture was compressed into tablets (700 mg per tablet) by a die having a 13 mmΦ flat bevel edge using tabletting machine. No darkishness by whittled powders or sticking of the mixture on the die was observed in the resulting tablets.

### Example 9

52.5 g of lansoprazole, 525 g of magnesium oxide (manufactured by Kyowa Kagaku Kogyo K.K., N grade), 761.3 g of magnesium hydroxide, 1352.8 g of D-mannitol and 70 g of aspartame were charged into a fluidized bed granulator, and an aqueous solution prepared by dispersing and dissolving 140 g of hydroxypropylcellulose, 1.75 g of yellow ferric oxide and 1.75 g of ferric oxide in 2376.5 g of purified water was sprayed, and these materials were granulated, and dried to obtain 2771.6 g of granules. 2573 g of the granules, 310 g of microcrystalline cellulose (Ceolus KG-801), 155 g of crospovidone and 62 g of magnesium stearate were mixed in a bag to obtain a mixture. The resultant mixture was compressed into tablets (1000 mg per tablet) by a die having a 16 mmΦ flat bevel edge using tabletting machine. No darkishness by whittled powders or sticking of the mixture on the die was observed in the resulting tablets.

### Example 10

300 g of lansoprazole, 500 g of magnesium oxide (manufactured by Kyowa Kagaku Kogyo K.K., N grade), 725 g of magnesium hydroxide, 1166.5 g of D-mannitol and 70 g of aspartame were charged into a fluidized bed granulator, and an aqueous solution prepared by dispersing and dissolving 140 g of hydroxypropylcellulose, 2.5 g of yellow ferric oxide and 1 g of ferric oxide in 2256.5 g of purified water was sprayed, and these materials were granulated, and dried to obtain 2783.0 g of granules.

2614.5 g of the granules, 315 g of microcrystalline cellulose (Ceolus KG-801), 157.5 g of crospovidone and 63 g of magnesium stearate were mixed in a bag to obtain a mixture. The resultant mixture was compressed into tablets (700 mg per tablet) by a die having a 13 mmΦ flat bevel edge using tabletting machine. No darkishness by whittled powders or sticking of the mixture on the die was observed in the resulting tablets.

### Example 11

210 g of lansoprazole, 525 g of magnesium oxide (manufactured by Kyowa Kagaku Kogyo K.K., N grade), 761.3 g of magnesium hydroxide, 1195.3 g of D-mannitol and 70 g of aspartame were charged into a fluidized bed granulator, and an aqueous solution prepared by dispersing and dissolving 140 g of hydroxypropylcellulose, 2.45 g of yellow ferric oxide and 1.05 g of ferric oxide in 2376.5 g of purified water was sprayed, and these materials were granulated, and dried to obtain 2823.7 g of granules.

2573 g of the granules, 310 g of microcrystalline cellulose (Ceolus KG-801), 155 g of crospovidone and 62 g of magnesium stearate were mixed in a bag to obtain a mixture. The resultant mixture was compressed into tablets (1000 mg per tablet) by a die having a 16 mmΦ flat bevel edge using tabletting machine. No darkishness by whittled powders or sticking of the mixture on the die was observed in the resulting tablets.

### Example 12

150 g of lansoprazole, 700 g of magnesium oxide (manufactured by Kyowa Kagaku Kogyo K.K., N grade), 435 g of magnesium hydroxide, 1406.5 g of D-mannitol and 70 g of aspartame were charged into a fluidized bed granulator, and an aqueous solution prepared by dispersing and dissolving 140 g of hydroxypropylcellulose and 3.5 g of yellow ferric oxide in 1906.5 g of purified water was sprayed, and these materials were granulated, and dried to obtain 2756.4 g of granules.

2614.5 g of the granules, 350 g of microcrystalline cellulose (Ceolus KG-801), 175 g of crospovidone and 70 g of magnesium stearate were mixed in a bag to obtain a mixture. The resultant mixture was compressed into tablets (700 mg per tablet) by a die having a 13 mmΦ flat bevel edge using a tabletting machine. No darkishness by whittled powders or sticking of the mixture on the die was observed in the resulting tablets.

### Experiment Example 1

Disintegration Test

The disintegration time was measured according to a method described in U.S. Pat. No. <701>Disintegration. Condition: purified water 1000 mL, no disk

The results are shown in Table 1.

TABLE 1

| | Example 1 | Example 2 | Example 3 |
|---|---|---|---|
| Average | 0.92 | 0.70 | 0.45 |
| disintegration | | | |
| time (min) | | | |

Measurement of pH Change

Test solution of 0.05 mol hydrochloric acid 100 mL (37° C.) was charged into a 100 mL beaker, and each one tablet obtained in example 1, example 2 and example 3 was added and a test was carried out under the condition of 100 revolutions per minute using a basket according to the dissolution test method of USP. pH change by time was measured.

As shown in Table 2, pH of the test solution increased quickly, and pH of 7 or more could be reached over 3 minutes.

TABLE 2

| | 1 min | 2 min | 3 min | 4 min | 5 min | 10 min |
|---|---|---|---|---|---|---|
| Example 1 | 1.42 | 3.12 | 7.63 | 8.83 | 9.04 | 9.15 |
| Example 2 | 2.01 | 6.77 | 7.97 | 8.46 | 8.64 | 8.85 |
| Example 3 | 3.08 | 6.99 | 7.49 | 7.72 | 7.83 | 8.06 |

Measurement of Dissolution Profile

One tablet obtained in example 1, example 2 or example 3, or one Takepron capsule (30 mg) filled with lansoprazole granules with an enteric coating was added to 900 mL of phosphate buffer solution having a pH of 6.8 at 37° C., and the amount of dissolved lansoprazole was measured under rotation at 75 rpm by the absorbancy at 286 nm in the ultraviolet range, and the dissolution ratio was calculated.

The results are shown in Table 3.

The dissolution profile was quick as compared with the dissolution of a capsule.

TABLE 3

| | 5 min | 10 min | 15 min | 20 min |
|---|---|---|---|---|
| Example 1 | 91.8% | 97.9% | 98.2% | 97.5% |
| Example 2 | 99.4% | 101.9% | 101.1% | 100.3% |
| Example 3 | 81.5% | 87.7% | 88.3% | 87.7% |
| Capsule | 38.1% | 94.2% | 96.8% | 97.7% |

### Experiment Example 2

Disintegration test

The disintegration time was measured according to a method described in USP <701>Disintegration. Condition: purified water 1000 mL, no disk

The results are shown in Table 4.

TABLE 4

| | Example 4 | Example 5 |
|---|---|---|
| disintegration | 1.25 | 1.28 |
| time (min) | | |

Measurement of dissolution profile

One tablet obtained in example 4 or example 5 was added to 900 mL of phosphate buffer solution having a pH of 6.8 at 37° C., and the amount of dissolved lansoprazole was measured by the absorbancy at 286 nm in the ultraviolet range under the same conditions as Experiment Example 1, and the dissolution ratio was calculated.

The dissolution profile was quick as compared with that of the above-mentioned Takepron capsule.

The results are shown in Table 5.

TABLE 5

| | 5 min | 10 min | 15 min | 20 min |
|---|---|---|---|---|
| Example 4 | 86.4% | 95.8% | 97.5% | 97.5% |
| Example 5 | 93.3% | 96.9% | 96.2% | 95.7% |

### Experiment Example 3

Disintegration Test

The disintegration time was measured according to a method described in USP <701>Disintegration. Condition: purified water 1000 mL, no disk

The results are shown in Table 6.

TABLE 6

| | Example 6 | Example 7 | Example 8 | Example 9 |
|---|---|---|---|---|
| disintegration | 1.8 | 1.98 | 1.95 | 1.98 |
| time (min) | | | | |

Measurement of Dissolution Profile

One tablet obtained in example 6, example 7, example 8 or example 9 was added to 900 mL of phosphate buffer solution having a pH of 6.8 at 37° C., and the amount of dissolved lansoprazole was measured by the absorbancy at 286 nm in the ultraviolet range under the same conditions as Experiment Example 1, and the dissolution ratio was calculated.

The dissolution profile was quick as compared with the dissolution of the capsule described above.

The results are shown in Table 7.

TABLE 7

| | 5 min | 10 min | 15 min | 20 min |
|---|---|---|---|---|
| Example 6 | 78.7% | 88.3% | 90.0% | 90.7% |
| Example 7 | 54.9% | 81.1% | 86.6% | 87.6% |
| Example 8 | 76.4% | 91.8% | 96.2% | 97.2% |
| Example 9 | 78.1% | 92.5% | 97.6% | 96.2% |

### Experiment Example 4

Disintegration test

The disintegration time was measured according to a method described in USP <701>Disintegration. Condition: purified water 1000 mL, no disk

The results are shown in Table 8.

TABLE 8

| | Example 10 | Example 11 | Example 12 |
|---|---|---|---|
| disintegration | 1.60 | 1.28 | 1.52 |
| time (min) | | | |

Measurement of Dissolution Profile

One tablet obtained in example 10, example 11 or example 12 was added to 900 mL of phosphate buffer solution having a pH of 6.8 at 37° C., and the amount of dissolved lansoprazole was measured by the absorbancy at 286 nm in the ultraviolet range under the same conditions as Experiment Example 1, and the dissolution ratio was calculated.

The results are shown in Table 9.

The dissolution profile was quick as compared with that of a capsule mentioned above.

TABLE 9

| | 5 min | 10 min | 15 min | 20 min |
|---|---|---|---|---|
| Example 10 | 73.7% | 82.4% | 83.7% | 83.7% |
| Example 11 | 59.1% | 72.6% | 76.4% | 78.8% |
| Example 12 | 85.4% | 95.2% | 96.7% | 97.9% |

Industrial Applicability

The medical solid preparation of the present invention can be obtained by a simple production method since no enteric coating is applied, though containing an acid labile active ingredient, for example, a benzimidazole compound which is a PPI. Further, since the initial dissolution of an active component from the preparation is quicker as compared with a preparation with an enteric coating, the initiation time of a pharmacological action can be shortened. Furthermore, since a metal oxide and metal hydroxide is mainly used for neutralization and stabilization in stomach, the generation of carbon dioxide gas which is generated in stomach by the administration of a preparation containing a bicarbonate or carbonate in a large amount can be suppressed, and therefore burp can be suppressed in the preparation.

1. A gastric disintegrable solid preparation comprising an acid labile active ingredient and at least one component selected from metal oxides and metal hydroxides:

2. A solid preparation according to claim 1, wherein the disintegration time is within 7 minutes.

3. A solid preparation according to claim 1, which is the preparation without enteric coating.

4. A solid preparation according to claim 1, which comprises further at least one component selected from carbonates of alkali earth metal and basic additives having high water-solubility.

5. A solid preparation according to claim 1, wherein an acid labile active ingredient is a proton pump inhibitor (PPI).

6. A solid preparation according to claim 5, wherein the PPI is a benzimidazole compound.

7. A solid preparation according to claim 6, wherein a benzimidazole compound is a compound represented by the formula (I): 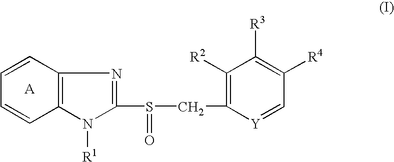
wherein ring A is an optionally substituted benzene ring, R¹ is hydrogen atom, an optionally substituted aralkyl group, acyl group or acyloxy group, R², R³ and R⁴ are the same or different and each represent a hydrogen atom, an optionally substituted alkyl group, an optionally substituted alkoxy group or an optionally substituted amino group, and Y represents a nitrogen atom or CH, or a salt thereof.

8. A solid preparation according to claim 6, wherein a benzimidazole compound is lansoprazole, omeprazole, rabeprazole or pantoprazole, or an optically active compound thereof.

9. A solid preparation according to claim 1, wherein the metal oxides and the metal hydroxides are those of which 1% aqueous solution or 1% aqueous suspension has a pH of 8.0 or more.

10. A solid preparation according to claim 1 which comprises at least one metal oxide selected from the group consisting of magnesium oxide, magnesium silicate, dry aluminum hydroxide gel and magnesium metasilicate aluminate.

11. A solid preparation according to claim 1 which comprises at least one metal hydroxide selected from the group consisting of magnesium hydroxide, aluminum hydroxide, synthetic Hydrotalcite, coprecipitate of aluminum hydroxide and magnesium hydroxide, coprecipitate of aluminum hydroxide, magnesium carbonate and calcium carbonate, and coprecipitate of aluminum hydroxide and sodium bicarbonate.

12. A solid preparation according to claim 4, wherein the carbonate of alkali earth metal is calcium carbonate or magnesium carbonate.

13. A solid preparation according to claim 4, wherein the basic additive having high water-solubility is trometamol, disodium succinate, sodium hydrogen phosphate, trisodium phosphate, dipotassium phosphate or L-arginine.

14. A solid preparation according to claim 1 which contains magnesium oxide.

15. A solid preparation according to claim 1 which contains magnesium hydroxide.

16. A solid preparation according to claim 1 which contains magnesium oxide and magnesium hydroxide.

17. A solid preparation according to claim 14, wherein the magnesium oxide is one obtained by calcination at a temperature ranging from about 500° C. to about 1000° C. and of purity higher than 95%.

18. A solid preparation according to claim 14, wherein the magnesium oxide has a BET specific surface area of about 10 m²/g to about 50 m²/g.

19. A solid preparation according to claim 6, which contains at least one component selected from metal oxides and metal hydroxides at a ratio of 0.1 to 1500 parts by weight relative to 1 part by weight of the benzimidazole compound.

20. A solid preparation according to claim 6, which contains at least one component selected from metal oxides and metal hydroxides together with a salt of alkali earth metal at a total ratio thereof of 0.1 to 1800 parts by weight relative to 1 part by weight of the benzimidazole compound.

21. A solid preparation according to claim 1, which is a tablet, a granule or a capsule.

22. A solid preparation according to claim 1, wherein a group containing an acid labile active ingredient and a group containing a metal oxide or a metal hydroxide but containing no active ingredient are separately compounded.

23. A solid preparation according to claim 4, wherein (1) a group containing both an active ingredient and at least one component selected from metal oxides, metal hydroxides, carbonates of alkali earth metal and basic additives having high water-solubility and (2) a group not containing an acid labile active ingredient but containing at least one component selected from metal oxides, metal hydroxides, carbonates of alkali earth metal and basic additives having high water-solubility are separately compounded.

24. A solid preparation according to claim 16, wherein the magnesium oxide is one obtained by calcination at a temperature ranging from about 500° C. to about 1000° C. and of purity higher than 95%.
